(19) United States
(12) Patent Application Publication
(10) Pub. No.: US 2024/0352399 A1
(43) Pub. Date: Oct. 24, 2024

(54) Method for isolating proteins and saccharides from yeast

(71) Applicant: Yeastup AG, Brugg (CH)

(72) Inventors: Wolfgang Riedl, Eimeldingen (DE); Rudi Verpoorten, Hagendorn-Cham (CH)

(73) Assignee: Yeastup AG, Brugg (CH)

(21) Appl. No.: 18/575,874

(22) PCT Filed: Jul. 12, 2022
(86) PCT No.: PCT/EP2022/069504

§ 371 Date: Jan. 1, 2024

(30) Foreign Application Priority Data
Jul. 12, 2021 (EP) 21185098.7

(51) Int. Cl. C12N 1/06; C12R 1/865

(52) U.S. Cl. CPC C12N 1/063; C12N 1/066; C12R 2001/865

(57) ABSTRACT

Method for digesting yeast, in particular brewer's yeast, for the isolation of proteins and saccharides comprising the steps:
a) Providing a yeast suspension, in particular a brewer's yeast suspension;
b) Physical digestion of the yeast in the yeast suspension;
c) Microfiltration of the digested yeast suspension from step b);
d) Ultrafiltration of the permeate of the microfiltration from step c) wherein a phase with proteins as the main solid component is separated as the retentate of the ultrafiltration;
e) Treating the retentate of the microfiltration from step c) with a protease under basic conditions and subsequently separating a phase with mannan as the main solid component, in particular by filtration and/or centrifugation;
f) treating the phase retained in step e) successively first at basic and then at acidic conditions and subsequently separating a phase with glucan as the main solid component, in particular by filtration and/or centrifugation.

### TECHNICAL FIELD

The invention relates to a method for digesting yeast, in particular brewer's yeast, for the isolation of proteins and saccharides.

### PRIOR ART

Yeasts are unicellular fungi that reproduce by sprouting or division and have always been of great technical importance. For example, yeasts are used in the production of beer, wine, spirits, food and therapeutic substances. Basically, there are several types of yeasts, with the so-called baker's or brewer's yeast (Saccharomyces cerevisiae) being one of the most commonly used yeasts. Accordingly, considerable quantities of yeast-containing and, in particular, brewer's yeast-containing by-products are produced.

Since the cell walls of yeasts in particular contain a number of technically useful biomolecules, such as proteins, glucans, mannans, chitins and lipids, yeast-containing waste is processed for the purpose of recovering these substances. In this process, the cell walls of the yeasts are partially or completely digested to release the substances of interest.

For example, EP 1 990 419 A1 (Tex-a-tec AG) describes a method for isolating glucan, protein, mannan and lipids from yeast, in which the yeast is treated with ultrasound in successive steps at increasing temperatures.

EP 2 272 876 A1 (Angel Yeast Co., Ltd.) discloses a process for extracting glucan and mannan from the cell wall of microorganisms, such as yeasts. The process includes the steps of: a) treating the cells of the microorganism with an alkaline protease and a mannanase; b) separating the mixture from step a) into a heavy phase and a light phase; c) drying the heavy phase obtained from step b) to obtain a glucan preparation; and d) drying the light phase obtained from step b) to obtain the mannan preparation. The glucan and mannan preparations are intended for therapeutic applications.

WO 2010/070207 A1 (Glykos Finland Oy) describes a process for preparing an immunostimulatory composition comprising a saccharide fraction, the process comprising the steps of hydrolyzing yeast cells and obtaining a soluble fraction. The resulting saccharide fraction can be incorporated as a food or beverage component or used as a pharmaceutical.

However, the methods known to date for isolating technically usable biomolecules from yeasts are not entirely convincing, especially in large-scale approaches. For example, the ratio of yield to effort, especially with regard to energy and time requirements, is unsatisfactory in many processes, which makes economic production very difficult.

Other processes are aimed at the selective recovery of individual components (e.g. glucan or mannan), while the remaining components (for example proteins) are ignored. This is disadvantageous in terms of maximizing the exploitation of yeast-containing by-products.

There is therefore still a need for improved solutions that at least partially or completely overcome the disadvantages mentioned.

### DISCLOSURE OF THE INVENTION

It is the object of the invention to provide an improved process for the recovery of biomolecules from yeast cells, in particular brewer's yeast cells. In particular, it should be possible to isolate several different biomolecules simultaneously with the highest possible yields and purities, especially preferably proteins, glucans and mannans. The process should also allow the biomolecules to be recovered as efficiently as possible in large-scale approaches. It is desirable that the process can be carried out with the lowest possible expenditure of energy and time.

The solution of the problem is defined by the features of claim 1. Accordingly, the core of the invention is a method for digesting yeast, in particular brewer's yeast, for the isolation of proteins and saccharides comprising the steps:
a) Providing a yeast suspension, in particular a brewer's yeast suspension;
b) Physical digestion of the yeast in the yeast suspension;
c) Microfiltration of the digested yeast suspension from step b);
d) Ultrafiltration of the permeate of the microfiltration from step c) wherein a phase with proteins as the main solid component is separated as the retentate of the ultrafiltration;
e) Treating the retentate of the microfiltration from step c) with a protease under basic conditions and subsequently separating a phase with mannan as the main solid component, in particular by filtration and/or centrifugation;
f) Treating the phase retained in step e) successively first at basic and then at acidic conditions and subsequently separating a phase with glucan as the main solid component, in particular by filtration and/or centrifugation.

Surprisingly, it has been shown that the combination of physical, chemical and biochemical treatment steps according to the invention allows good yields with relatively low energy and time requirements. This applies to all products, i.e. to proteins, glucans and mannans, and yields for these substances of up to about 40% each can be achieved at high purity.

Furthermore, the process is suitable for large-scale approaches. Several tens of thousands of tons of yeast suspension per year or several thousand kilograms of yeast suspension per hour can be processed without any problems.

“Glucans” are oligo- or polysaccharides composed of D-glucose molecules linked by glycosidic bonds. Glucans as found in yeast cells are typically beta-glucans, which have a β-glycosidic bond, in particular β-1,3-glucan. “Mannans” are polysaccharides that are polymers of the sugar mannose. Mannans found in yeast typically have an α (1-6)-linked backbone and α (1-2)- and α (1-3)-linked side chains that average about two sugar units in length. “Microfiltration” means a process for filtration through a filter medium, in particular a membrane, with a pore size of 0.1 μm. In contrast, the pore size in ultrafiltration is less than 0.1 μm.

Unless otherwise stated, the “solid component” refers in each case to the dry mass of the respective component. The term “main solid constituent” refers to the constituent of the solids with the largest proportion by weight.

The yeast suspension provided in step a) is in particular a yeast suspension stored at a temperature of 0-15° C., preferably 2-12° C., in particular 3-7° C. or 5° C. and/or a pasteurized yeast suspension. This ensures that the suspension contains as few undesirable by-products as possible.

In principle, however, the yeast suspension provided in step a) can also be a yeast suspension stored at a different temperature, in particular room temperature, and not pasteurized.

The yeast suspension is in particular an aqueous yeast suspension. Water is present as a continuous liquid phase of the yeast suspension.

The yeast suspension is in particular a brewer's yeast suspension, which preferably contains used brewer's yeast. Such brewer's yeast suspensions occur, for example, as by-products in beer production and can be used directly in the method according to the invention.

The yeast suspension preferably has a solids content of 5-30% by weight, in particular 7-20% by weight, based on the total weight of the yeast suspension. This has proved to be particularly suitable for the method according to the invention. If necessary, the solids content of the yeast suspension can be adjusted with liquid, in particular water, within the desired range.

In step a), the yeast suspension has in particular a temperature of 0-15° C., preferably 2-12° C., in particular 3-7° C. or 5° C. This ensures that the composition of the suspension does not change further, e.g. due to active yeast cells, or that the formation of undesirable by-products is reduced.

Further preferably, step b), and optionally also steps c) and d), is also carried out at a temperature of 0-15° C., preferably 2-12° C., more particularly 3-7° C. or 5°° C. This further reduces the formation of undesired by-products.

In principle, however, the yeast suspension in steps a), b), c) and/or d) can also be at a different temperature, for example room temperature.

According to an advantageous embodiment, the prepared yeast suspension is subjected to a washing process before step b), in which at least part of the liquid phase of the yeast suspension, in particular at least 50% by weight, in particular at least 60% by weight, of the liquid phase of the yeast suspension, is exchanged by a further liquid or an exchange liquid, in particular water. The washing process preferably takes place in a centrifuge or a drum filter.

If necessary, the washing process can reduce the proportion of dissolved and unwanted components in the liquid, thus improving the purity and quality of the substances to be isolated. However, a washing process is optional and can also be omitted.

The physical digestion in step b) is carried out in particular by electric fields, in a homogenizer or a grinding media mill, especially in a ball mill.

The homogenizer is in particular a high-pressure homogenizer, which is operated at a pressure of more than 1,000 bar, in particular more than 1,200 bar.

In a grinding media mill, material to be ground and freely movable grinding media, in particular balls, are circulated in a process chamber. This results in impacts between the grinding media, the walls of the process chamber and the material to be ground. The material to be ground is comminuted primarily by crushing.

Digestion with a grinding media mill, in particular a ball mill, has proven to be particularly advantageous in the present case, since the proteins to be isolated can be released gently but effectively. This applies in particular to brewer's yeast suspensions. However, other physical digestion methods can also be used for special applications.

The physical digestion by means of electric fields in step b) can be carried out in particular by means of pulsed electric fields (also referred to as

PEF or “Pulsed Electric Fields”). This digestion technique is known to the skilled person in other contexts per se.

In pulsed electric field treatment, the yeast suspension is placed in particular between two electrodes and treated with pulsed electric fields. The pulsed electric fields are characterized in particular by high field strengths and a short duration. Reversible or irreversible pore formation takes place in the cell membranes, whereby intracellular components are released and/or extracellular substances can penetrate into the cells.

In particular, the following parameters have been found to be suitable for controlling the digestion of yeast in the yeast suspension: the electric field strength [kV/cm], the input temperature of the yeast suspension [° C.] and the specific energy input [kJ/L]. Energy input is understood as the energy required for the method in kilojoules per liter of yeast suspension. Furthermore, the energy input can be controlled by the frequency of the pulses [Hz], which in turn depends on the flow rate and is automatically controlled in known systems.

Preferably, the electric field strength is 1-30 kV/cm, in particular 5-24 kV/cm.

Preferably, the specific energy input is 1-180 KJ/L, in particular 3-120 kJ/L.

The input temperature of the yeast suspension is preferably 0-15° C., preferably 2-12° C., more particularly 3-7° C. or 5° C.

With such parameters, the yeast in the yeast suspension can be effectively digested.

Equipment for performing physical cell disruption using pulsed electric fields is commercially available from various suppliers, such as the PEF Pilot Dual system (Elea-Technology; Germany).

The treatment by pulsed electric fields is preferably followed by a mechanical post-treatment. This is thus preferably carried out between steps b) and c).

The mechanical post-treatment can be carried out in particular by means of a mixer, preferably with high shear forces. A rotor-stator mixer, in particular an inline rotor-stator mixer, is particularly suitable. In an inline rotor-stator mixer, the rotor and stator are typically contained in a housing with an inlet at one end and an outlet at the other end. The yeast suspension to be treated can thereby be drawn through the mixer in a continuous stream.

In particular, the shear rate during mechanical post-treatment with a mixer is selected such that the yeast cells are further disrupted. This allows the intracellular components (proteins), which may not yet have been completely released after application of the pulsed electric fields, to be released. The high shear forces additionally “squeeze out” the already opened yeast cells and intracellular components, in particular the proteins, can escape from the cells.

Microfiltration in step c) is preferably carried out with a filter medium with a pore size of 0. 1-0.5 μm, preferably 0. 1-0.2 μm, in particular 0.1 μm. This has proven to be particularly preferred in order to achieve a relative enrichment of the proteins to be isolated in the permeate after physical digestion in relation to the solids content and at the same time an enrichment of biomass residues, e.g. cell fragments and fats, in the retentate.

In particular, the microfiltration in step c) is carried out as a diafiltration, in which an exchange liquid, in particular water, is preferably continuously supplied on the feed side. In other words, during diafiltration, the liquid phase of the digested yeast suspension, which is discharged as permeate, is preferably continuously replaced on the feed side at least partially, in particular completely, by an exchange liquid, in particular by water.

In particular, the digested yeast suspension is circulated from a feed bin. By regulating the flow downstream of the filter medium, a transmembrane pressure can be set which acts as a driving force in the diafiltration.

The diafiltration is preferably operated in a closed circuit so that the discharge of the permeate leads to the afterflow of the exchange solvent, in particular in such a way that the circulating volume remains constant during the diafiltration.

Specifically, the microfiltration in step c) is preferably carried out in such a way that a proportion of proteins in the permeate of the microfiltration from step c), based on the total weight of solids in the permeate, is preferably at least 50% by weight, more preferably 50-80% by weight, in particular 60-70% by weight.

A total solids content in the permeate of the microfiltration in step c) is preferably 1-10% by weight, in particular 3-7% by weight, based on the total weight of the permeate.

Further preferably, the microfiltration in step c) is carried out in such a way that a proportion of proteins in the retentate of the microfiltration from step c) relative to the total weight of solids in the retentate is less than 50% by weight, preferably less than 40% by weight, in particular less than 35% by weight.

A total solids content in the retentate of the microfiltration in step c) is preferably 20-50% by weight, in particular 25-30% by weight, based on the total weight of the retentate.

According to another possible embodiment, the digested yeast suspension is treated with an oxidizing agent, in particular ozone (O₃) and/or hydrogen peroxide (H₂O₂), before microfiltration step c). In particular, this can prevent chemical reactions that lead to undesirable by-products and/or changes in taste. However, treatment with an oxidizing agent is optional. Further, the digested yeast suspension can be treated enzymatically prior to microfiltration step c). This can increase the efficiency and yield of the process if required. However, enzymatic treatment before microfiltration step c) is optional.

The ultrafiltration in step d) is preferably carried out with a filter medium having a pore size of less than 90 nm and/or an exclusion limit (nominal molecular weight cut-off) of 1-100 kDa, preferably 10-50 kDa, in particular 15-25 kDa, more specifically 20 kDa. The exclusion limit is defined as the minimum molecular mass of molecules which are retained by the filter medium, in particular the membrane, to 90%.

In particular, the ultrafiltration in step d) is carried out as a diafiltration, in which an exchange liquid, in particular water, is preferably continuously supplied on the feed side. In other words, in the diafiltration in step d), the liquid phase of the permeate of the microfiltration from step c), which is discharged as permeate in the diafiltration, is preferably continuously replaced on the feed side at least partially, in particular completely, by an exchange liquid, in particular by water.

The permeate of the microfiltration from step c), which forms the feed in step d), is circulated in particular from a feed bin. By regulating the flow after the filter medium, a transmembrane pressure can in turn be set, which acts as the driving force of the diafiltration in step d).

The diafiltration in step d) is preferably operated in a closed circuit so that the discharge of the permeate leads to the afterflow of the exchange liquid, in particular so that the circulating volume remains constant during the diafiltration.

The ultrafiltration and diafiltration in step d) can be used to selectively increase the relative solid content of the protein in the retentate.

Preferably, the ultrafiltration in step d) is carried out in such a way that a proportion of the proteins in the phase separated in step d) with proteins as the main solid component is at least 75% by weight, in particular at least 85% by weight, preferably at least 90% by weight, based on the weight of the solids.

A total solids content in the phase separated in step d) is preferably 0.5-7% by weight, in particular 1-3% by weight, based on the total weight of the phase separated in step d).

Specifically, both the microfiltration in step c) and the ultrafiltration in step d) are performed as diafiltration.

Particularly preferably, the phase separated in step d) with proteins as the main solid component is dried after step d), in particular spray-dried. As a result, the protein is obtainable as a powdered product with high purity. Corresponding devices and processes for spray drying are known per se to the person skilled in the art.

Particularly preferably, the solids content, especially the protein content, is concentrated before spray drying, in particular to a solids content of at least 50% by weight, in particular at least 60% by weight, preferably at least 70% by weight. The concentration is carried out in particular in an evaporator, which partially evaporates the liquid phase. Corresponding equipment and processes for concentration are known to the person skilled in the art.

Optionally, the phase separated in step d) with proteins as the main solid component after step d) can be treated with an oxidizing agent, in particular ozone and/or hydrogen peroxide, and/or contacted with an adsorbent material, e.g. activated carbon. In this way, in particular, the product quality of the proteins can be improved. For example, undesirable discolorations and/or flavors can be chemically decomposed and/or removed.

If spray drying is performed, treatment with the oxidant and/or contacting with the adsorbent material preferably occurs prior to spray drying and, if such occurs, prior to concentration.

Treatment with an oxidizing agent is particularly preferably carried out with a membrane contactor. In a membrane contactor, the fluids undergoing mass transfer are guided past each other separated by a porous membrane. In the present case, the fluids are the phase separated in step d) with proteins as the main solid component and the oxidant. The oxidant passes through the membrane in the membrane contactor into the phase with proteins as the main solid component separated in step d). The use of a membrane contactor has proven to be particularly advantageous and efficient in comparison with other contactors. This is particularly true since the pressure loss and energy requirement in the membrane contactor are relatively low, but the oxidizing agent can still exert its effect very effectively.

In step e), a protease is used. Proteases are enzymes that can hydrolytically cleave proteins. Proteases are also called proteolytic enzymes.

Preferably, in step e), the protease is used at a level of 0.0001-10% by weight, in particular 0.001-5% by weight, more specifically 0.01-1% by weight or 0.05-0.5% by weight, based on the solids content of the retentate of the microfiltration from step c).

The protease is preferably an alkaline protease, in particular a serine protease, especially preferably a subtilisin.

Serine proteases are a subfamily of proteases that have the amino acid serine in their active site.

Subtilisin-type proteases (subtilases, subtilopeptidases, EC 3. 4. 21. 62) are classified as serine proteases on the basis of their catalytically active amino acids. They are naturally produced and secreted by microorganisms, especially by Bacillus species. They act as non-specific endopeptidases, i.e. they hydrolyze arbitrary acid amide bonds located inside peptides or proteins. Their pH optimum is usually in the clearly alkaline range.

Proteases, and in particular the specific proteases mentioned, have proven to be extremely advantageous in the present method. In particular, the protease can selectively release mannan, which can be separated in the form of a liquid phase with mannan as the main solid component.

Specifically, the process is carried out without the addition of mannanase or in the absence of mannanase. Mannanase is an enzyme that degrades mannans.

The treatment in step e) is preferably carried out at a pH of 7.5-13, preferably 8-11, in particular 8.5-10.5, and/or at a temperature of 40-80° C., preferably 50-70° C., in particular 55-65° C. The pH value is preferably adjusted to the protease used.

In particular, in step e) the pH is adjusted by adding a base, preferably a base with a pKg value of less than 4.75, in particular less than 1. The pKB value stands for the negative decadic logarithm of the base constant KB. Particularly preferred is an inorganic base, especially NaOH. Such bases have proven to be particularly suitable.

The base is preferably used in diluted form, e.g. at a level of 30-60% by weight in water.

In particular, the treatment in step e) lasts 10 min to 18 hours, especially 3-12 hours. This allows the yield to be optimized.

In a further embodiment, a pasty fraction can be processed with a small amount of liquid in step e). Thus, depending on the composition of the yeast suspension and reaction control, the enzymatic step can be optimized with regard to yield.

In step f), the retained phase is preferably treated in a first time interval, preferably for 1-5 hours, at a pH of 7.5-13, preferably 8-11, in particular 8.5-10.5, and/or at a temperature of 40-95° C., preferably 65-85° C., in particular 75-85° C. Subsequently, the retained phase is preferably treated in a second time interval, preferably for 0.5-2 hours, at a pH of 2-6.5, preferably 3-5, in particular 3.5-4.5, and/or at a temperature of 50-95° C., preferably 80-95° C., in particular 80-90° C.

By the “phase retained in step e)” mentioned in step f) is meant in particular that phase which remains after separation of the phase with mannan as the main solid constituent under step e). In particular, the “phase retained in step e)” thus corresponds to the retentate from step c) from which the phase with mannan as the main solid constituent was separated.

If the phase with mannan as the main solid component is separated in step e), for example by filtration, the “phase retained in step e)” referred to in step f) is in particular the retentate from step e). In this case, the phase with mannan as the main solid component in step e) forms the permeate, which is separated.

Particularly preferably, in step f) the pH value in the first time interval is selected such that it corresponds to the pH value present during the treatment in step e).

It is further preferred if in step f) the temperature in the first time interval is higher than the temperature present during the treatment in step e).

Preferably, in step f) the temperature in the first time interval is lower than in the second time interval and/or the first time interval is longer than the second time interval.

In step f), if necessary, the pH is adjusted by adding a base, preferably NaOH, and/or by adding an acid, preferably H₂SO₄.

Preferably, the base is a base as described above in step e). Particularly preferably, the base in step f) is the same base as in step e).

The acid is in particular an acid with a pKₛ value of less than 4.75, in particular less than 1. The pKₛ value stands for the negative decadic logarithm of the acid constant Kₛ. Particularly preferred is a mineral acid, especially H₂SO₄.

The acid is preferably used in diluted form, e.g. at a level of 30-60% by weight in water.

These aforementioned measures, especially in combination, have proven to be particularly advantageous in terms of the efficiency of the process.

The separation of the phase with mannan as the main solid component in step e) is preferably carried out by filtration, in particular a combined microfiltration and ultrafiltration. Similarly, the separation of the phase with glucan as the main solid component in step f) is preferably carried out by filtration, in particular a combined microfiltration and ultrafiltration.

The phase remaining after separation of the phase with mannan in step e), is further treated in step f) in particular as the “retained phase”.

Microfiltration is preferably carried out in each case with a filter medium having a pore size of 0.1-0.5 μm, in particular 0. 1-0.2 μm, preferably 0. 1 μm. Ultrafiltration is preferably carried out in each case with a filter medium having a pore size of less than 90 nm and/or an exclusion limit (nominal molecular weight cut-off) of 1-100 kDa, preferably 10-50 kDa, in particular 15-25 kDa, especially 20 kDa.

The microfiltration in step e) and/or the ultrafiltration in step f) are preferably carried out as diafiltration, in particular in the same way as described for steps c) and d).

Here, in step e), in particular the phase with mannan as the main solid component is removed in the form of the permeate of the microfiltration, fed to the ultrafiltration and the relative proportion of mannan in the retentate of the ultrafiltration is increased.

In step f), in particular, the phase with glucan as the main solid component is removed in the form of the permeate of the microfiltration, fed to the ultrafiltration and the relative proportion of glucan in the retentate of the ultrafiltration is increased.

During diafiltration in steps e) and/or f), the liquids are circulated in particular from the reaction vessel in which steps e) and/or f) take place, with exchange liquid preferably being continuously supplied on the feed side.

According to a further advantageous embodiment, the separation in steps e) and/or f) is carried out by a drum filter and/or a centrifuge. This in particular in step f).

Accordingly, in a particular embodiment, the separation in step e) is carried out by a combined microfiltration and ultrafiltration as described above, and the separation in step f) is carried out by a drum filter and/or a centrifuge, in particular a centrifuge.

Preferably, the phase separated in step e) with mannan as the main solid component is dried after step e), in particular spray-dried, and/or the phase separated in step f) with glucan as the main solid component is dried after step f), in particular spray-dried. As a result, both mannan and glucan are obtainable as powdered products with high purity.

Preferably, before spray-drying, the solids content, in particular the mannan and/or glucan content, is concentrated, in particular with an evaporator.

Optionally, the phase separated in step e) with mannan as the main solid constituent can be treated after step e) with an oxidizing agent, in particular ozone and/or hydrogen peroxide, and/or contacted with an adsorber material, e.g. activated carbon.

Similarly, the phase separated in step f) with glucan as the main solid constituent may optionally be treated after step f) with an oxidizing agent, in particular ozone and/or hydrogen peroxide, and/or contacted with an adsorber material, e.g. activated carbon.

This can improve the product quality of mannan and/or glucan, as with protein separation.

If spray drying is carried out after steps e) and/or f), treatment with the oxidizing agent and/or contacting with the adsorbent material is preferably carried out before spray drying and, if such is carried out, before concentration.

The treatment with an oxidizing agent is particularly preferably carried out with a membrane contactor. Advantages in this respect have been described above in connection with the protein stream.

Further advantageous embodiments and combinations of features of the invention result from the following detailed description and the totality of the patent claims.

### BRIEF DESCRIPTION OF THE DRAWINGS

FIGS. 1 and 2, used to explain the embodiment example, show the process flow diagram of a process according to the invention for digesting brewer's yeast for the purpose of isolating proteins (P), mannan (M) and glucan (G).

In principle, the same parts are given the same reference signs in the figures.

### WAYS TO CARRY OUT THE INVENTION

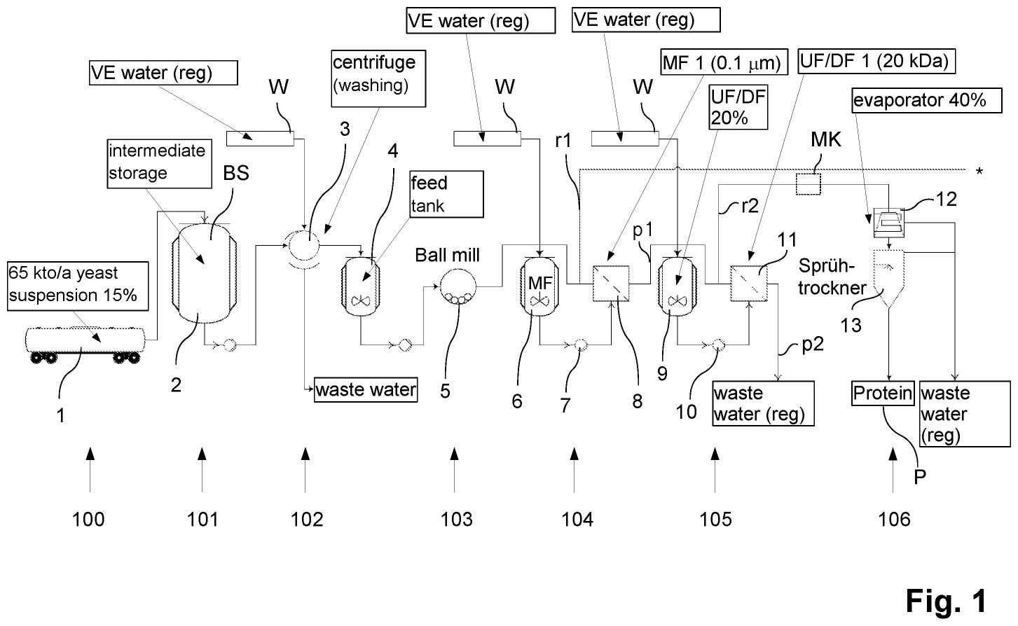

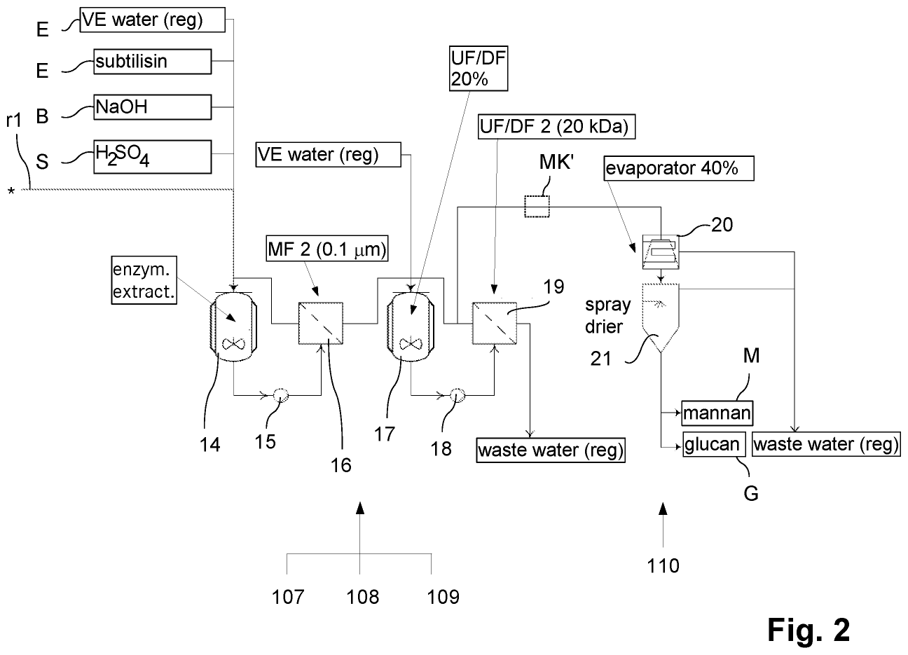

FIGS. 1 and 2 show the process flow diagram of a method according to the invention for digesting brewer's yeast for the purpose of isolating proteins (P), mannan (M) and glucan (G).

In a first step 100, a cooled aqueous brewer's yeast suspension BS (temperature=5° C.; solids concentration=15% by weight) is delivered by a tank truck 1 to be temporarily stored in a storage tank 2 at 5° C. in the subsequent step 101.

The brewer's yeast suspension BS is then subjected to a washing process 102 in a centrifuge 3. In this process, the liquid phase of the brewer's yeast suspension BS is replaced by water W and fed to a feed tank 4.

From the feed tank 4, the washed brewer's yeast suspension is then fed into a ball mill 5, where the brewer's yeast cells in the suspension are physically digested in the next process step 103.

The digested brewer's yeast suspension is then subjected to a microfiltration step 104. The microfiltration is carried out as a diafiltration with a filter medium with a pore size of 0. 1 μm. The digested brewer's yeast suspension is circulated from a feed bin 6 through the microfilter 8 by a pump 7, with water W being continuously supplied on the feed side as an exchange liquid. The transmembrane pressure, which can be regulated by the permeate outflow, is approx. 1.5 bar. A proportion of proteins in the permeate p1 of the microfiltration, based on the total weight of solids in the permeate p1, is, for example, 60-70% by weight.

The permeate p1 of the microfiltration step 104 is then subjected to an ultrafiltration step 105. The ultrafiltration is carried out as a diafiltration using a filter medium with an exclusion limit (nominal molecular weight cut-off) of 20 kDa. In this process, the permeate p1 is circulated from another feed bin 9 via the ultrafilter 11 using a pump 10, with water W being continuously supplied on the feed side as an exchange liquid. The transmembrane pressure, which can be regulated by the permeate outflow, is approximately 2 bar. The ultrafiltration step 105 increases the relative solids content of the protein in the retentate r2, so that a solids content of the proteins relative to the total solids content in the remaining retentate r2 is about 90% by weight.

The permeate p2 of the ultrafiltration step 105 is discharged as wastewater (this can be recycled if required).

The retentate r2 of the ultrafiltration step 105 with proteins as the main solid component is subjected to a drying process 106, the retentate r2 being fed to a spray drying device 13 after passing through an evaporator 12. This results in the protein as a powdered product P.

The yield of protein over the entire process is about 38% by weight based on the content of available proteins in the brewer's yeast suspension BS.

The retentate r1 from the microfiltration process 104 undergoes further stepwise enzymatic and chemical treatment, which is shown in FIG. 2. FIG. 2 connects with the location marked with an asterisk (*) to the location marked with an asterisk in FIG. 1.

In a proteolytic digestion step 107, the retentate r1 is treated in a reactor 14, for example, for 8 hours with 0.2% by weight (based on the solids content of the retentate r1) of a protease E in the form of subtilisin. The treatment is carried out at a pH of about 9.5, the pH being adjusted by adding base B in the form of a 45% aqueous solution of NaOH and kept constant during the treatment with protease.

Proteolytic digestion selectively releases mannan, which is subsequently separated via microfiltration and downstream ultrafiltration in the form of an aqueous phase with mannan as the main solid component. Both microfiltration and ultrafiltration are carried out as diafiltrations. During microfiltration, the liquid to be filtered is circulated by a pump 15 from reactor 14 over microfiltration filter 16 (pore size: 0.1 μm), with exchange liquid being continuously supplied on the feed side. The permeate from the microfiltration is then fed to the feed bin 17 of the ultrafiltration stage, from which the liquid is circulated by a pump 18 over the ultrafilter 19 (exclusion limit: 20 kDa). The ultrafiltration increases the relative solid content of mannan in the retentate of the ultrafiltration stage, so that a solid content of mannan relative to the total solid content in the remaining retentate is about 65% by weight. The permeate from the ultrafiltration stage is discharged as wastewater (this can be reprocessed if required).

The retentate of the ultrafiltration stage with mannan as the main solid component is subjected to a drying process 110, whereby the retentate is fed to a spray drying device 21 after passing through an evaporator 20. This results in the mannan as a powdered product M.

The yield of mannan over the total process is about 39% by weight based on the content of available mannan in the brewer's yeast suspension BS.

Then the phase retained in reactor 14 is heated to a temperature of about 80° C. and subjected to a basic extraction 108 at a pH of 9.5 in an initial time interval of about 3 hours. The pH is kept constant by controlled addition of dilute NaOH.

Subsequently, the temperature is increased again to approx. 85° C. and the pH is lowered to a value of 4 by adding 50% H₂SO4 in water. Thereupon, the phase retained in the reactor is subjected to acid extraction 109 in a time interval of 1 hour.

After basic and acidic extraction, a liquid phase with glucan as the main component is separated by diafiltration via the two filter stages (microfilter 16 and ultrafilter 18) in the same way as for mannan. As a result, a solid content of glucan of approx. 72% by weight is achieved in relation to the total solid content in the retentate of the diafiltration stage.

The retentate of the ultrafiltration stage with glucan as the main solid component is also subjected to a drying process 110, the retentate being fed to the spray drying device 21 after passing through the evaporator 20. This results in the glucan as a powdered product G.

The yield of glucan over the entire process is about 42% by weight based on the content of available glucan in the brewer's yeast suspension BS.

However, the invention is not limited to the embodiment example shown. This can thus be modified as desired within the scope of the invention.

For example, other yeast suspensions may be used instead of a brewer's yeast suspension. Likewise, non-mandatory process steps, such as the washing process 102, can be omitted. Furthermore, cell digestion can be performed by pulsed electric fields instead of using a ball mill 5. It is also possible, among other things, to separate the mannan and/or the glucan using a drum filter or a centrifuge instead of the filter stages described (microfilter 16 and ultrafilter 18).

Furthermore, a membrane contactor MK or MK′ can optionally be provided upstream of the evaporator 12 and/or upstream of the evaporator 20. This allows the corresponding phases to be treated with an oxidizing agent, for example ozone and/or hydrogen peroxide, prior to spray drying.

In summary, it can be stated that a novel and particularly advantageous method for the digestion of yeast, in particular brewer's yeast, for the isolation of proteins and saccharides has been provided, which is particularly suitable for large-scale approaches.

We claim:

1-18. (canceled)

19. A method for digesting yeast for the isolation of proteins and saccharides comprising the steps:
a) providing a yeast suspension;
b) physical digestion of the yeast in the yeast suspension;
c) microfiltration of the digested yeast suspension from step b);
d) ultrafiltration of the permeate of the microfiltration from step c) wherein a phase with proteins as the main solid component is separated as the retentate of the ultrafiltration;
e) treating the retentate of the microfiltration from step c) with a protease under basic conditions and subsequently separating a phase with mannan as the main solid component; and
f) treating the phase retained in step e) successively first at basic and then at acidic conditions and subsequently separating a phase with glucan as the main solid component.

20. The method according to claim 19, wherein the yeast suspension provided in step a) has a solids content of 5-30% by weight, based on the total weight of the yeast suspension.

21. The method according to claim 19, wherein the yeast suspension provided in step a) has a temperature of 0-15° C.

22. The method according to claim 19, wherein the yeast suspension is subjected to a washing process before step b), in which at least part of the liquid phase of the yeast suspension is replaced by another liquid.

23. The method according to claim 19, wherein the physical digestion in step b) is carried out in a homogenizer or a grinding media mill.

24. The method according to claim 19, wherein the physical digestion in step b) is performed by pulsed electric fields.

25. The method according to claim 24, wherein the electric field strength is 1-30 kV/cm and/or the specific energy input is 1-180 KJ/L.

26. The method according to claim 19, wherein the microfiltration in step c) is carried out with a filter medium having a pore size of 0.1-0.5 μm, and wherein the ultrafiltration in step d) is carried out with a filter medium having a pore size of less than 90 nm and/or an exclusion limit of 1-100 kDa.

27. The method according to claim 19, wherein the microfiltration in step c) and/or the ultrafiltration in step d) is carried out as diafiltration wherein water is continuously supplied on the feed side.

28. The method according to claim 19, wherein the phase separated in step d) with proteins as the main solid component is dried after step d).

29. The method according to claim 19, wherein in step e) protease is used in a proportion of 0.0001-10% by weight, based on the solids content of the retentate of the microfiltration from step c).

30. The method according to claim 19, wherein the treatment with protease in step e) lasts 10 min to 18 hours.

31. The method according to claim 19, wherein in step f) the retained phase is treated in a first time interval for 1-5 hours at a pH of 7.5-13 and a temperature of 40-95°° C., and subsequently treated in a second time interval for 0.5-2 hours at a pH of 2-6.5 and a temperature of 50-95° C.

32. The method according to claim 31, wherein in step f) the pH in the first time interval is equal to the pH present during the treatment in step e), and wherein in step f) the temperature in the first time interval is lower than in the second time interval and/or the first time interval is longer than the second time interval.

33. The method according to claim 19, wherein the separation of the phase with mannan as the main solid component in step e) and/or the separation of a phase with glucan as the main solid component in step f) is carried out by filtration.

34. The method according to claim 33, wherein the microfiltration is carried out with a filter medium having a pore size of 0.1-0.5 μm, and wherein the ultrafiltration is carried out with a filter medium having a pore size of less than 90 nm and/or an exclusion limit of 1-100 kDa.

35. The method according to claim 33, wherein the microfiltration in step e) and/or the ultrafiltration in step f) is carried out as diafiltration.

36. The method according to claim 19, wherein the phase separated in step e) with mannan as the main solids component after step e) is dried, and/or wherein the phase separated after step f) with glucan as the main solids component after step f) is dried.

37. The method according to claim 19, wherein the yeast is brewer's yeast.

38. The method according to claim 29, wherein the protease is a subtilisin and wherein the treatment in step e) is carried out at a pH of 7.5-13 and at a temperature of 40-80° C.
